(19) United States
(12) Patent Application Publication
(10) Pub. No.: US 2006/0088558 A1
(43) Pub. Date: Apr. 27, 2006

(54) Vitamin E TPGS fluid concentrate comprising a low percentage of water

(76) Inventors: Robert Jandzinski, Johnson City, TN (US); Andreas M. Papas, Kingsport, TN (US); Konstantinos A. Papas, Jonesborough, TN (US); Constantinos Neophytou, Nicosia (CY)

Correspondence Address: DONNA J. RUSSELL, 1492 ANTHONY WAY, MT. JULIET, TN, 37122, US

(21) Appl. No.: 11/258,783

(22) Filed: Oct. 26, 2005

Related U.S. Application Data
(60) Provisional application No. 60/622,179, filed on Oct. 26, 2004.

(51) Int. Cl. A61K 38/43; A61K 9/00; A61K 47/00; A61K 31/355

(52) U.S. Cl. 424/400; 424/439; 424/94.1; 514/458

(57) ABSTRACT

A liquid Vitamin E TPGS concentrate composition is provided, the concentrate having a significantly higher percentage of TPGS per unit volume than traditional liquid TPGS formulations while providing the TPGS in a liquid form that can be used in softgels or mixed with water to produce desired concentrations for commercial use in supplements, beverages, pharmaceutical preparations, etc.

This application claims the benefit of priority of U.S. Provisional Patent Application No. 60/622,179 filed Oct. 26, 2004.

### FIELD OF THE INVENTION

The invention relates to compositions and methods for preparing compositions comprising liquid and water-soluble concentrates of lipophilic compounds. More specifically, the invention relates to compositions and method for preparing compositions comprising fluid concentrates of vitamin E TPGS formulations.

### BACKGROUND OF THE INVENTION

TPGS, or alpha-tocopheryl polyethylene glycol 1000 succinate, is a solubilizing agent and emulsifier prepared by esterifying d-alpha-tocopheryl succinate with polyethylene glycol 1000. At room temperature, TPGS is a waxy compound. It has both hydrophilic and lipophilic properties, however, and is miscible with water, forming solutions in water at concentrations of up to twenty percent (20%) TPGS (wt/wt). These solutions are often used to provide a water-soluble form of Vitamin E used in supplements, especially for those with fat malabsorption syndromes such as chronic childhood cholestasis. It is also used as an absorption and bioavailability enhancer for certain water-insoluble drugs such as the HIV protease inhibitor amprenavir, and fat-soluble vitamins such as vitamin D.

TPGS is generally provided as a solid form that is melted into a liquid state by heating to above 40° C. and stirring to ensure homogeneity. Weighed portions of the melted liquid TPGS are poured slowly into measured volumes of boiling sterile water, which are constantly stirred for 1 to 2 hours while cooling down to room temperature. Concentrations of TPGS in water below 20 percent by weight are free-flowing liquids, while those above 20 percent TPGS do not stay in solution and solidify in a time range from seconds to 2 weeks, depending on the ratio of the components. When TPGS concentration is increased to above 20 wt %, higher viscosity liquid crystalline phases begin to form. Providing TPGS in its solid form, however, requires that additional steps be taken by the customer in order to produce a liquid form for use. It is a pale yellow, waxy solid substance, and the waxy nature of the solid form makes it more difficult to measure desired amounts for preparation of the 20% solution than would a granular or liquid form.

Neither the waxy solid TPGS nor the liquid form, because of its high water content requirement, are suitable for preparation of many dosage forms such as softgels and other pharmaceutical, nutraceutical, and nutritional preparations that cannot be formed of ingredients having a percentage of water of, for example, more than ten percent.

It would be of great benefit for the preparation of TPGS compositions for use in supplements, beverages, pharmaceutical preparations and other products to provide fluid concentrates of TPGS that could be incorporated into formulations requiring the use of lower percentages of water and could more easily be measured and mixed than the solid form.

### SUMMARY OF THE INVENTION

The presenting invention provides a composition comprising Vitamin E TPGS and at least one lipophile in an amount effective to promote the formation of a fluid composition when water is added to the composition in an amount comprising not more than about 15% by weight of the total weight of an admixture of water and the composition. In one embodiment of the invention, water is present in amounts of about 2 to about 15 percent by weight of the admixture. In some embodiments, water is present in amounts of about 3 to about 11 percent by weight of the admixture.

In some embodiments of the invention the at least one lipophile comprises at least about 25% by weight of the admixture, and in certain embodiments the at least one lipophile is chosen from among the group consisting of tocopherols, tocotrienols, carotenoids, coenzyme Q10, omega fatty acids, and mixtures thereof.

The invention also provides a fluid composition comprising Vitamin E TPGS, at least one lipophile; and water in an amount not to exceed about 15 percent by weight of the composition.

Furthermore, the invention provides a method for forming a fluid Vitamin E TPGS concentrate comprising less than about 80 percent water (wt/wt), the method comprising admixing Vitamin E TPGS with at least one lipophile and water, the water comprising about 2 to about 15 percent by weight of the composition and the at least one lipophile comprising at least about 25% by weight of the concentrate.

In some embodiments of the method the water comprises about 3 to about 11 percent by weight of the composition. In some embodiments of the method the lipophile is chosen from among the group consisting of tocopherols, tocotrienols, carotenoids, coenzyme Q10, omega fatty acids, and mixtures thereof.

The invention provides a fluid TPGS concentrate comprising about 3 to about 15 percent by weight of water.

The invention also provides TPGS compositions comprising gelatinous, pudding, semi-solid and fluid consistencies comprising about 3 to about 15 percent by weight of water.

### DETAILED DESCRIPTION

The present invention provides a TPGS “concentrate” that can provide significantly higher percentage concentrations of TPGS per unit volume than have previously described in the art while providing the TPGS in a liquid form that can be used in softgels, or can form, for example, pastes, puddings, soft gelatinous strips or other oral delivery formulations or can be admixed with water to produce the desired concentrations for use in liquids such as beverages (including juices, carbonated beverages such as soft drinks, dairy products, protein or diet etc.) The inventors have discovered that the addition of a lipophilic agent to a mixture of water and TPGS can provide a liquid formulation with percentages of water in the formulation that are significantly lower (2-15%, for example) than the 80% water formulations that must be used to maintain a TPGS/water composition in its fluid state. Unlike the TPGS/water compositions comprising no more than about twenty percent (20%) (wt/wt) TPGS to maintain the composition in its fluid state, TPGS/lipophile/water compositions can be maintained in the non-solid state when the amount of water in the composition is from about 2 to about 15 percent water, and more preferably about 3 to about 10 percent water, providing a fluid TPGS concentrate. This TPGS/lipophile/water mixture is a free flowing liquid, a syrup, a paste, or other non-solid form that does not solidify during storage.

As used herein, the term “TPGS” is used interchangeably with the term vitamin E TPGS, and describes compounds having the chemical formula C₃₃ O₅ H₅₄ (CH₂ CH₂ O)n, where “n” represents the number of polyethylene oxide moieties attached to the acid group of crystalline d-alpha tocopheryl succinate. Vitamin E TPGS is commercially available from Eastman Chemical Company, Kingsport, Tenn., USA. According to Eastman product literature, Vitamin E TPGS is shelf-stable both in its pure form and after blending into liquids. Formulators can store Vitamin E TPGS at room temperature for months. When provided in its pure form, it is a solid that can be melted into a liquid state by heating to above 40° C. and stirring to ensure homogeneity. Weighed portions of the solid form are poured slowly into measured volumes of boiling sterile water, which are constantly stirred for 1 to 2 hours while cooling down to room temperature.

The term “fluid,” as used herein, encompasses non-solid forms of a combination comprising TPGS and water. Such forms can include, for example, liquids, gels, syrups, puddings and pastes. The specific form of the composition may be varied by increasing or decreasing the amount of water while remaining within the range of about 2 to about 15%. A liquid, syrupy, or slightly gelatinous composition, for example, may be incorporated into softgels, provided generally that the concentration of water in the composition is low enough to maintain the integrity of the softgel coating (e.g., no higher than 10% for most commercially-available gelatin coating compositions).

Compositions comprising TPGS and a lipophile such as one or more vitamins, antioxidants, or therapeutic agents that may be less soluble or bioavailable unless they are combined with TPGS may be provided in a single dose pack, for example, as a paste or pudding. Compositions containing TPGS, water, and one or more lipophiles may also form syrups that can be administered in single dose packs or in multi-dose packages that can provide more consistent dosing of specific amounts of the desired lipophile. Liquid compositions can also be added to beverages, or can be provided to the consumer as an add-in for nutritional drinks, for example, to provide Vitamin E TPGS in conjunction with desired vitamins, antioxidants, pharmaceutical agents, etc.

The present invention provides fluid lipophilic emulsions using TPGS at significantly lower ratios of water to TPGS than had previously described. One benefit of the invention, for example, is that a concentrate as provided by the invention can be used to form liquid TPGS compositions or add TPGS to liquid compositions such as beverages, pharmaceutical syrups, etc., without the need to melt the TPGS, measure it, maintain the temperature while stirring for long periods of time, etc., at the site, such as a product manufacturing site, where the TPGS formulation will be added to the liquid composition. Because of its solubility in aqueous compositions, it can be added to beverage formulations, pharmaceutical preparations, and other compositions. Unlike oil-soluble vitamin E additives that require additional steps, and perhaps additional equipment, for addition to an aqueous product, it can be blended into beverages or other aqueous compositions as a final step.

In one embodiment, the composition of the invention is prepared by adding one or more lipophiles, heated to about 45° C. to about 50° C., to melted TPGS (heated to about 45° C. to about 50° C.) with continuous stirring. To provide a fluid concentrate, the percentage by weight of lipophile in the TPGS/lipophile mixture should be at least about 25%, although the amount of lipophile required may be less, depending upon the properties of the lipophile chosen. The range of percentage by weight of water should be from at least about 3 to about 15%, but variations in the upper limit may be observed with different lipophilic agents. For a particular lipophile, for example, a percentage of water above 12%, may provide a composition that has a more paste-like consistency that could be used, for example, for encapsulation in softgels or for preparation of foil pacs that serve as a paste delivery system for medications or nutrients, while a lower percentage by weight of water may provide a composition that has a more syrupy or liquid consistency.

Suitable lipophiles, or lipophilic agents, can be chosen by one of skill in the art. Lipophiles appropriate for use in compositions of the present invention include, but are not limited to, tocopherols, tocotrienols, carotenoids, coenzyme Q10, omega fatty acids and mixtures thereof. Lipophilic agents can provide additional desirable nutritional or pharmaceutical benefit in combination with the benefit provided by the TPGS ingredient within the composition, and can be chosen accordingly.

In one embodiment, a TPGS concentrate as provided by the invention can be reconstituted to a final concentration of approximately 80% water, for example, by diluting the concentrate by the addition of the desired amount of heated water to the heated concentrate with continuous stirring. It is preferable to add water to the concentrate, rather than adding concentrate to water, to avoid thickening that may occur when the percentage of water in the concentrate increases to above 15%.

It is to be understood that compositions of the present invention may be combined with, and may comprise, flavorings, excipients, stabilizers, and other agents known to those of skill in the art of formulation of food, beverage, and pharmaceutical formulation. The invention may be further described by means of the following non-limiting examples.

### EXAMPLES

Vitamin E TPGS was obtained from Eastman Chemical Company (Kingsport, Tenn., USA). MTS-90 is a product of Archer Daniels Midland and is an oil of high concentrations (92.5%) of mixed tocopherols. Of the tocopherols, d-gamma-tocopherol is of the highest concentration (64%), followed by d-delta-tocopherol (16%), d-alpha-tocopherol (11%), and d-beta-tocopherol (1%). Mixed Tocotrienols 92 is a product of Eisai Food and Chemical Company (Tokyo, Japan) and is an oil of high concentrations (98%) of mixed tocopherols and mixed tocotrienols. The mixed tocotrienols content is 70% and the total content of mixed tocotrienols and mixed tocopherols is 98%. Nutriene™ is a product of Eastman Chemical Company and is an oil mixture of tocotrienols and tocopherols. The mixed tocotrienol content is 15% and the mixed tocopherol content is 20%.

Preparation of TPGS/Lipophile Concentrates

Mixture A was produced by combining 146.1 grams of TPGS with lipophile provided as 56.25 grams of MTS90, and 22.5 grams of Nutriene™. TPGS was melted at 50° C. before mixing, and the other two ingredients were heated to 50° C. before mixing. The mixture was maintained at 45° C. This composition was admixed with water, slowly, with stirring, in the amounts shown in Table 1 to produce liquids as described in Table 1. Water was provided at 90° C.

TABLE 1

| | | Percentage of water | |
|---|---|---|---|
| | | In resulting | |
| Mixture A | Water | concentrate | Description |
| 48.5 g | 1.5 g | 3% | Clear, viscous liquid |
| 46.5 g | 3.5 g | 7% | Clear, viscous liquid |
| 44.5 g | 5.5 g | 11% | Very thick, somewhat |
| | | | cloudy, viscous liquid |
| 42.5 g | 7.5 g | 15% | Non-flowing, somewhat |
| | | | cloudy gel |

Mixture B was produced by combining 146.1 grams of TPGS with lipophile provided by 78.8 grams of Nutriene. TPGS was melted at 50° C. before mixing, and the other two ingredients were heated to 50° C. before mixing. The mixture was maintained at 45° C. This composition was admixed with water, slowly, with stirring, in the amounts shown in Table 2 to produce liquids as described in Table 2. Water was provided at 90° C.

TABLE 2

| | | Percentage of water | |
|---|---|---|---|
| | | In resulting | |
| Mixture B | Water | concentrate | Description |
| 48.5 g | 1.5 g | 3% | Cloudy, thick liquid |
| 46.5 g | 3.5 g | 7% | Cloudy, thick liquid |
| 44.5 g | 5.5 g | 11% | Non-flowing, somewhat |
| | | | cloudy gel |
| 42.5 g | 7.5 g | 15% | Non-flowing, somewhat |
| | | | cloudy gel |

Mixture C was produced by combining 146.1 grams of TPGS with lipophile provided as 56.3 grams of MTS90, and 22.5 grams of Eisai 92 (Eisai 5 Company, Ltd., Tokyo, Japan). TPGS was melted at 50° C. before mixing, and the other two ingredients were heated to 50° C. before mixing. The mixture was maintained at 45° C. This composition was admixed with water, slowly, with stirring, in the amounts shown in Table 3 to produce liquids as described in Table 3. Water was provided at 90° C.

TABLE 3

| | | Percentage of water | |
|---|---|---|---|
| | | In resulting | |
| Mixture B | Water | concentrate | Description |
| 48.5 g | 1.5 g | 3% | Clear, viscous liquid |
| 46.5 g | 3.5 g | 7% | Clear, viscous liquid |
| 44.5 g | 5.5 g | 11% | Clear, viscous liquid |
| 42.5 g | 7.5 g | 15% | Non-flowing, somewhat |
| | | | cloudy gel |

TPGS/Beta-Carotene Formulations

In a flask, 245 g of Eastman Vitamin E TPGS-1000 was stirred under nitrogen at 65-70° C. To the TPGS was added (slowly, over a 2-hour period) 120.8 g 30% beta-carotene in vegetable oil (Hoffman-LaRoche, Basel, Switzerland), followed by the addition over 20 minutes of 34.0 g potassium sorbate and 0.080 g linoleic acid, to provide a total weight of approximately 400 grams. This was used as a stock solution and maintained at 70° C. to prepare Samples 1 through 8 as described below:

Sample 1: 49 g stock solution combined with 1 g H₂O, stirred at 70° C. for 1 hour and then cooled to room temperature.

Sample 2: 48 g stock solution combined with 2 g H₂O, stirred at 70° C. for 1 hour and then cooled to room temperature.

Sample 3: 47 g stock solution combined with 3 g H₂O, stirred at 70° C. for 1 hour and then cooled to room temperature.

Sample 4: 46 g stock solution combined with 4 g H₂O, stirred at 70° C. for 1 hour and then cooled to room temperature.

Sample 5: 44 g stock solution combined with 6 g H₂O, stirred at 70° C. for 1 hour and then cooled to room temperature.

Sample 6: 40 g stock solution combined with 10 g H₂O, stirred at 70° C. for 1 hour and then cooled to room temperature.

Sample 7: 30 g stock solution combined with 20 g H₂O, stirred at 70° C. for 1 hour and then cooled to room temperature.

Sample 8: 5 g stock solution combined with 45 g H₂O, stirred at 70° C. for 1 hour and then cooled to room temperature.

Samples were observed and phase and consistency and observations were recorded. When cooled to room temperature, the stock solution solidified. Samples 1 through 4 remained fluid and had a syrupy consistency after cooling to room temperature. Samples 5 through 7 solidified at room temperature and at 70° C. Sample 8 was fluid, separating slightly into two phases when allowed to sit, but easily returning to one phase when shaken or stirred.

What is claimed is:

1. A composition comprising
Vitamin E TPGS;
at least one lipophile in an amount effective to promote the formation of a fluid when water is added to the composition in an amount comprising not more than about 15% by weight of the total weight of an admixture of water and the composition.

2. The composition of claim 1 wherein water is present in amounts of about 2 to about 15 percent by weight of the admixture.

3. The fluid composition of claim 1 wherein water is present in amounts of about 3 to about 11 percent by weight of the admixture.

4. The fluid composition of claim 1 wherein the at least one lipophile comprises at least about 25% by weight of the admixture.

5. A fluid composition as in claim 1 wherein the at least one lipophile is chosen from among the group consisting of tocopherols, tocotrienols, carotenoids, coenzyme Q10, and mixtures thereof.

6. A fluid composition comprising
Vitamin E TPGS;
at least one lipophile; and
water in an amount not to exceed about 15 percent by weight of the composition.

7. A method for forming a flowable Vitamin E TPGS concentrate comprising less than about 80 percent water (wt/wt), the method comprising admixing Vitamin E TPGS with at least one lipophile and water, the water comprising about 2 to about 15 percent by weight of the composition and the at least one lipophile comprising at least about 25% by weight of the concentrate.

8. The method of claim 7 wherein the water comprises about 3 to about 11 percent by weight of the composition.

9. The method of claim 7 wherein the lipophile is chosen from among the group consisting of tocopherols, tocotrienols, carotenoids, coenzyme Q10, and mixtures thereof.

10. A composition comprising a fluid TPGS concentrate comprising about 3 to about 15 percent by weight of water.

11. The composition of claim 10 wherein the concentrate comprises a liquid, a syrup, a gel, a pudding, or a paste.
